(19) United States
(12) Patent Application Publication
(10) Pub. No.: US 2008/0167268 A1
(43) Pub. Date: Jul. 10, 2008

(54) Particulate beta-glucan compositions for regulating dendritic cells

(76) Inventor: Jun Yan, Louisville, KY (US)

Correspondence Address: Bioplymer Engineering, Inc.;dba Biothera, Inc., c/o Intellevate, P.O. Box 52050, Minneapolis, MN, 55402, US

(21) Appl. No.: 11/899,021

(22) Filed: Sep. 4, 2007

Related U.S. Application Data
(60) Provisional application No. 60/841,795, filed on Sep. 1, 2006.

(51) Int. Cl. A61K 31/716

(52) U.S. Cl. 514/54

(57) ABSTRACT

Particulate β-glucan is administered at either a low concentration range or a high concentration range to affect cytokine secretion. At the high concentration range, particulate β-glucan induces secretion of inflammatory cytokines that enhance antigen presentation by dendritic cells and T cell expansion. Conversely, at the low concentration range, particulate β-glucan induces secretion of anti-inflammatory cytokines leading to immunological tolerance.

This application claims the benefit of U.S. Ser. No. 60/841,795 entitled PARTICULATE β-GLUCAN COMPOSITIONS FOR REGULATING DENDRITIC CELLS, filed on Sep. 1, 2006.

### BACKGROUND OF THE INVENTION

The present invention relates to compositions for treating anti-proliferative disorders. More particularly, the present invention relates to treating anti-proliferative disorders by stimulation of dendritic cells with β-glucan.

Adaptive immunity is specific and encompasses the second line of host defense against invasion of foreign organisms. Antigen presentation forms the basis of this type of immune response in order to launch an appropriate antibody response.

The most efficient antigen-presenting cells (APCs) are mature dendritic cells (DCs). The ability of DCs to regulate immunity is dependent on DC maturation. A variety of factors can induce maturation following antigen uptake and processing including whole bacterial or bacterial-derived antigens, inflammatory cytokines, engagement of specific cell surface receptors and viral products. Phenotypic and functional changes during the DC maturation process involve redistribution of major histocompatibility complex (MHC) molecules from intracellular compartments to the cell surface, down-regulation of antigen internalization, increased surface expression of costimulatory molecules, morphological changes and cytoskeletal reorganization, secretion of chemokines, cytokines and proteases and surface expression of adhesion molecules and chemokine receptors.

Immature DCs capture antigens by phagocytosis, macropinocytosis or through interaction with specific receptors and endocytosis. Intracellular proteins are then involved with uptake and processing. Following processing, antigenic peptides are presented via MHC molecules to CD4⁺, CD8⁺ or memory T cells.

DCs process either exogenous or endogenous antigens and present these antigens in the context of either MHC class I or II molecules. Antigen transport to the cell surface via MHC class II coincides with increased costimulatory molecule expression, which amplify T cell receptor signaling and elevate T cell activation. Antigens complexed with MHC class I molecules are presented to CD8-expressing T cells to produce cytotoxic T cells.

Upon activation, the DCs migrate to T cell-rich areas of lymphoid organs. The migration process is regulated by chemokine/chemokine receptor interactions and aided by various proteases in conjunction with their corresponding receptors. Another set of receptors on the surface of DCs mediate contact between DCs and T cells allowing the DCs to screen T cells for an appropriate T cell receptor (TCR).

Once an antigen has been recognized, an army of immune cells is created that is specifically designed to attack that antigen. Adaptive immunity also includes a “memory” that makes future responses against a specific antigen more efficient.

β-glucan is a complex carbohydrate derived from sources including yeast and other fungi, bacteria and cereal grains. The potential antitumor activity of β-glucans has been under extensive investigation. The effectiveness of various glucan preparations has differed in their ability to elicit various cellular responses, particularly cytokine expression and production, and in their activity against specific tumors.

β-glucans are generally thought to operate through the innate immune system, which includes complement proteins, macrophages, neutrophils and natural killer (NK) cells. However, it would also be advantageous to affect APCs for treatment of various proliferative and autoimmune disorders.

### SUMMARY OF THE INVENTION

Particulate β-glucan is administered at either a low concentration range or a high concentration range to affect cytokine secretion. At the high concentration range, particulate β-glucan induces secretion of inflammatory cytokines that enhance antigen presentation by dendritic cells and T cell expansion. Conversely, at the low concentration range, particulate β-glucan induces secretion of anti-inflammatory cytokines leading to immunological tolerance.

### BRIEF DESCRIPTION OF THE DRAWINGS

FIG. 1 shows graphical representations of flow cytometry results indicating the presence of OVA, Thy1.1 and MUC1 surface markers on Lewis Lung Carcinoma cells.

FIG. 2 shows flow cytometry plots of Staurosporine-induced apoptosis of Lewis Lung Carcinoma cells.

FIG. 3 shows flow cytometry plots of bone marrow-derived dendritic cell uptake of apoptotic Lewis Lung Carcinoma cells.

FIG. 4 shows flow cytometry histograms of surface marker expression levels on bone marrow-derived dendritic cells stimulated with particulate β-glucan.

FIG. 5 shows flow cytometry plots indicating cytokine profiles of bone marrow-derived dendritic cells stimulated with particulate β-glucan.

FIG. 6 graphically shows antigen-specific T cell responses stimulated by particulate β-glucan.

FIG. 7 shows flow cytometry plots of in vivo apoptotic tumor cell uptake by dendritic cells.

FIG. 8 shows flow cytometry histograms that quantitate in vivo apoptotic tumor cell uptake by dendritic cells.

FIG. 9 shows surface marker expression of dendritic cells stimulated in vivo with particulate β-glucan.

### DETAILED DESCRIPTION OF THE INVENTION

While immunotherapy with monoclonal antibodies (MAbs) has proved to be a promising approach in the treatment of cancer and other proliferative disorders, its success has not been as great as initially hoped. Studies utilizing β-glucan in combination with tumor-specific MAbs have significantly increased their effectiveness by eliciting components of the innate immune system. However, particulate β-glucan compositions of the present invention treat proliferative disorders, such as cancer, by stimulating the adaptive immune system. The system and experiments described below form the basis of the present invention.

A lung cancer model was used in this dendritic cell application. Lewis-lung tumor cells (LLC) were transfected with OVA and MUC1 genes, which create specific markers on the tumor cells. Ovalbumin (OVA) acts as a surrogate marker specific for OVA specific T (OTII) cells. Mucin MUC1 is expressed on the tumor cell surface. Anti-mucin Abs and β-glucan effectively destroy tumors that express mucin.

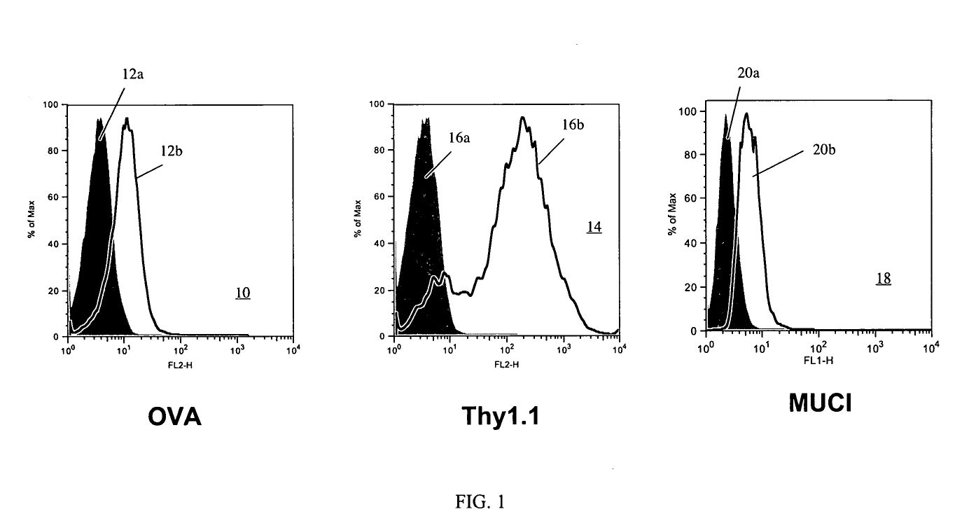

Results of successful transfection of OVA and MUC1 into LLCs are shown in FIG. 1. Graph 10, which shows OVA expression, includes histograms 12a and 12b. Histogram 12a is the isotype control Ab staining, while histogram 12b is OVA specific staining. The shift of histogram 12b relative to histogram 12a indicates OVA expression on the LLC.

Graph 14 indicates the presence of another LLC surface marker, Thy1.1. Histograms 16a and 16b represent isotype control and Thy1.1 specific Ab staining, respectively.

Graph 18 includes histograms 20a and 20b, which represent isotype control and MUC1 specific Ab staining. Again, the shift of histogram 20b relative to 20a indicates expression of MUC1.

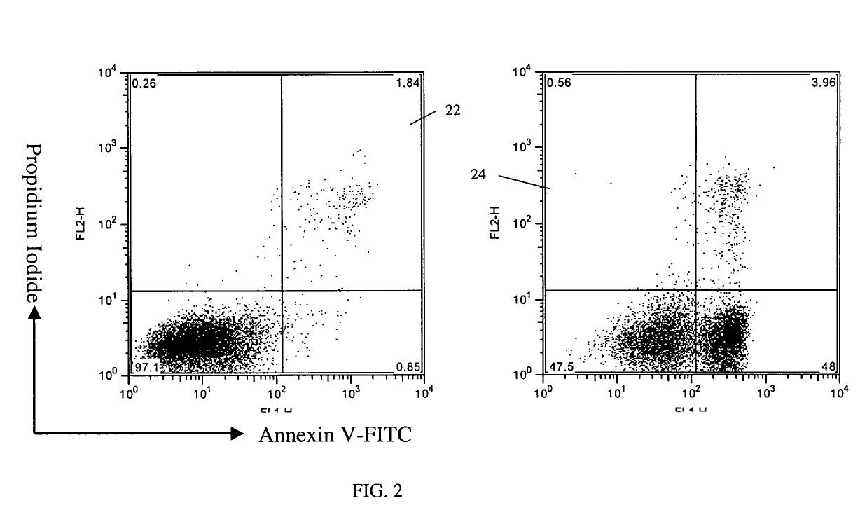

The capacity to induce apoptosis of transfected LLCs was tested, and the results are shown in FIG. 2. Transfected LLCs were either left untreated (plot 22) or treated (plot 24) with Staurosporine, which induces apoptosis. Cells from both groups were stained with propidium iodide (PI) and annexin V-FITC and analyzed by flow cytometry.

As shown in plot 22, most of the untreated, transfected LLCs are in the lower left quadrant and did not stain with either PI or annexin V-FITC. The double negative results indicate that most of the untreated population consists of live cells.

Conversely, in plot 24, the population of LLCs were treated with Staurosporine. A significant number of these cells have positively stained with annexin V-FITC and shifted to the lower right quadrant. The shifted cells are apoptotic. Therefore, Staurosporine is able to induce apoptosis of the transfected LLCs.

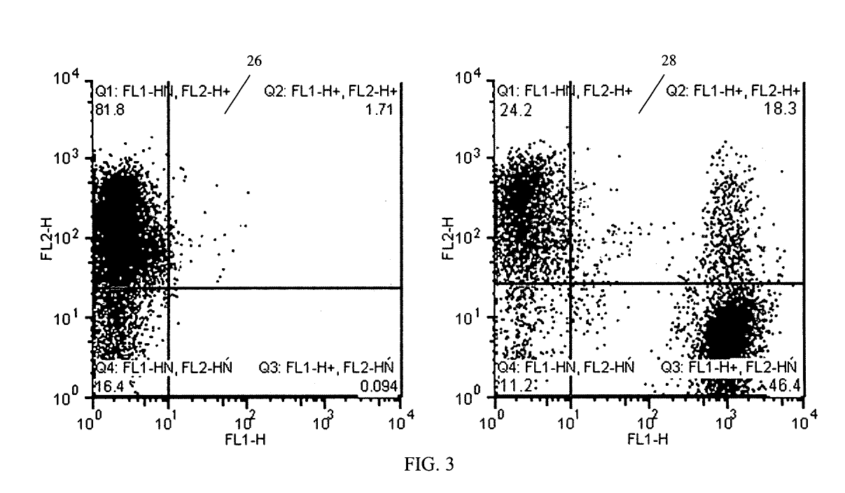

Next, the ability of FLT3L bone marrow-derived dendritic cells (BMDCs) to uptake apoptotic LLCs was tested. FLT3L is a growth factor for hematopoietic progenitors and induces hematopoietic progenitor and stem cell mobilization in vivo. Results are shown in FIG. 3.

The population of BMDCs in plot 26 was not exposed to stained apoptotic LLCs, and, as expected, most of the BMDCs are in the upper left quadrant (single stained). The population of BMDCs in plot 28 was exposed to stained apoptotic LLCs, and a subpopulation of the BMDCs shifted to the upper right quadrant (doubled stained). Thus, some BMDCs were able to uptake the apoptotic LLCs.

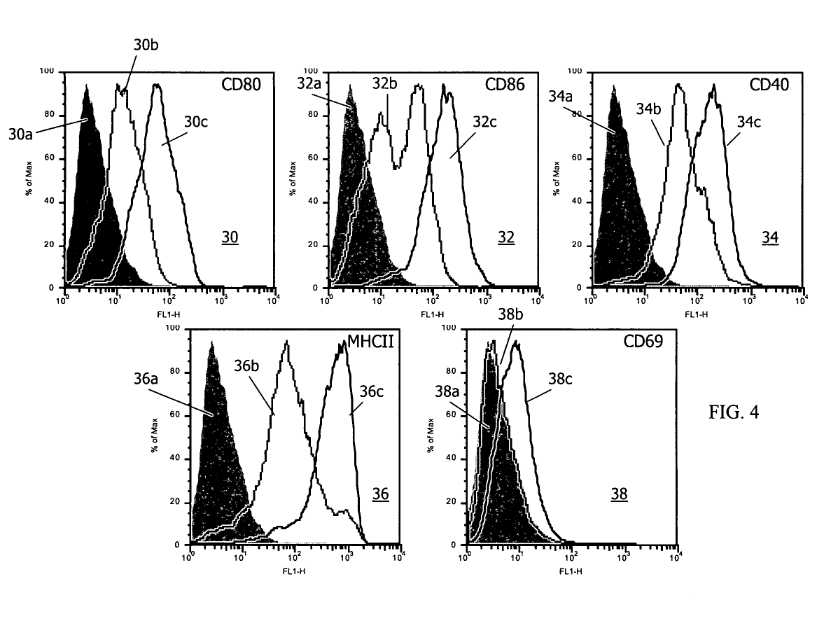

Since it was determined that BMDCs were able to uptake apoptotic LLCs, the next experiment was designed to show whether or not stimulation of BMDCs with particulate β-glucan resulted in DC maturation signals. FLT3L BMDCs were incubated either with or without 100 μg/ml particulate β-glucan. Sets of BMDCs were then stained with anti-CD80, anti-CD86, anti-CD40, anti-MHC class II or anti-CD69. Expression of CD80, CD86, CD40, MHC class II and CD69 indicate DC activation and maturation. The results are shown in FIG. 4.

Graphs 30, 32, 34, 36 and 38 represent expression of CD80, CD86, CD40, MHC class II and CD69, respectively. Each graph includes three histograms representing an isotype Ab control, Ab stained unstimulated BMDCs and Ab stained BMDCs stimulated with particulate β-glucan.

With respect to graph 30, histogram 30a (filled) represents the isotype Ab control. Histograms 30b (discontinuous) and 30c (continuous) represent anti-CD80 staining of unstimulated and stimulated BMDCs, respectively. Analogous histograms are included in each of the remaining graphs 32-38. The shift of histograms 30c, 32c, 34c, 36c and 38c relative to histograms 30b, 32b, 34b, 36b and 38b, respectively, indicate increased expression of each of the tested surface markers. These results suggest that particulate β-glucan activates DC for maturation.

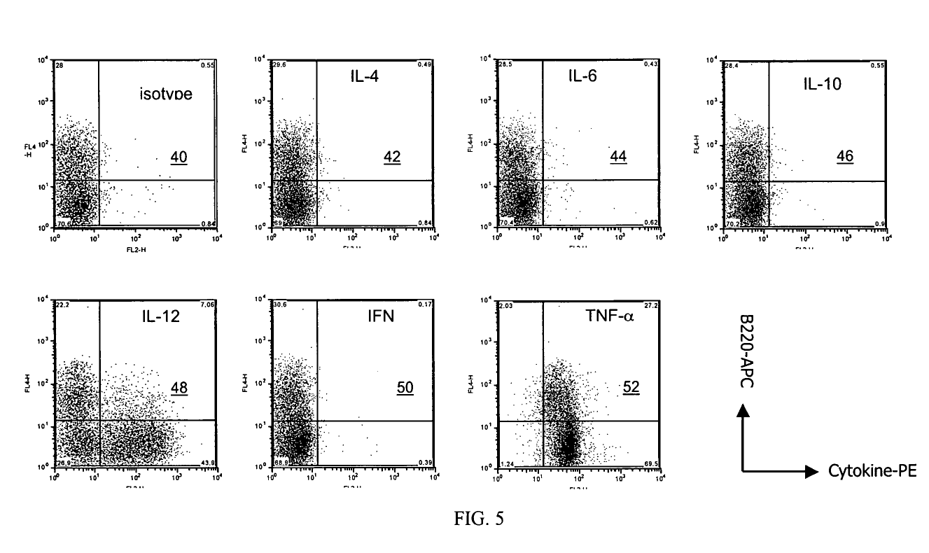

The cytokine profile of particulate β-glucan-stimulated BMDCs was then determined. BMDCs were stimulated with 100 μg/ml particulate β-glucan and cytokine production was correlated to corresponding intracellular mRNA levels. Results are shown in FIG. 5.

Plot 40 shows cells labeled with an isotype control. Plots 42, 44, 46, 48, 50 and 52 represent IL-4, IL-6, IL-10, IL-12, IFN-γ and TNF-α production, respectively. Plots 48 and 52 indicate that the stimulated BMDCs produce IL-12 and TNF-α, which are inflammatory cytokines. IL-6 is also an inflammatory cytokine, however, a profile shift would only be expected with macrophages. These results are further evidence that particulate β-glucan activates DC maturation.

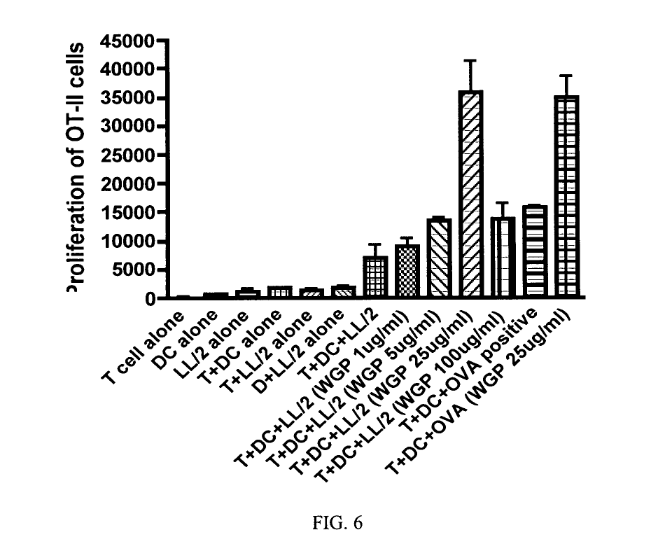

Next, the ability of particulate β-glucan to enhance specific T cell responses was tested. OTII cells are T cells activated in response to the Ova peptide. These cells were incubated in the presence of various combinations of DCs, LLCs, particulate β-glucan and Ova peptide, and the relative levels of T cell proliferation were determined. Proliferation was measured by isotope incorporation. The results are shown in the bar graph of FIG. 6.

The last bar, which represents T cell proliferation in the presence of DCs, Ova peptide and 25 μg/ml particulate β-glucan, is a positive control. As is evident from the graph, a comparable amount of T cell proliferation occurred when OTII cells were incubated with DCs, LLCs and 25 μg/ml particulate β-glucan. Lesser amounts of particulate β-glucan also resulted in antigen-specific T cell proliferation but to a lesser extent. These results show that particulate β-glucan enhances antigen-specific T cell responses.

The experiments described above showed uptake of apoptotic tumors cells by DCs and enhanced T cell proliferation with particulate β-glucan in vitro. The next experiments were carried out to determine whether the same held true in vivo.

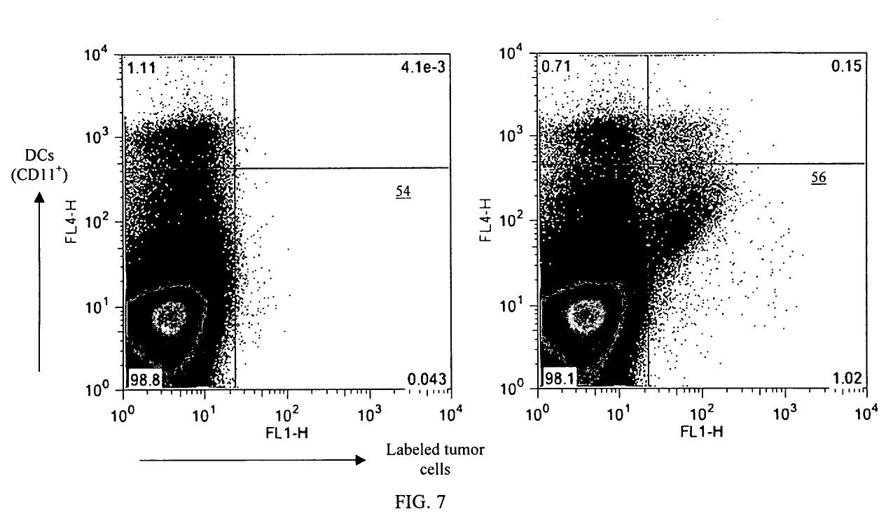

Mice were injected intravenously with tumor cells labeled with a non-specific fluorescent surface dye. Spleens were harvested at a later time and the dispersed cells were labeled with Abs to identify the CD11c⁺ cells. CD11c⁺/tumor cells positive cells were identified by flow cytometry. The results are shown in FIG. 7.

Plot 54 of FIG. 7 is the flow cytometry results of a negative control where mice were injected with a solution that did not contain tumor cells. The population of DCs (CD11c⁺) from the dispersed spleen cells is represented in the upper left quadrant of the plot. The results of spleen cells from test mice (i.e. injected with tumor cells) are shown in plot 56. CD11c+DCs that also contained labeled tumor cells have shifted to the upper right quadrant. These results clearly indicate that DCs uptake apoptotic tumor cells in vivo.

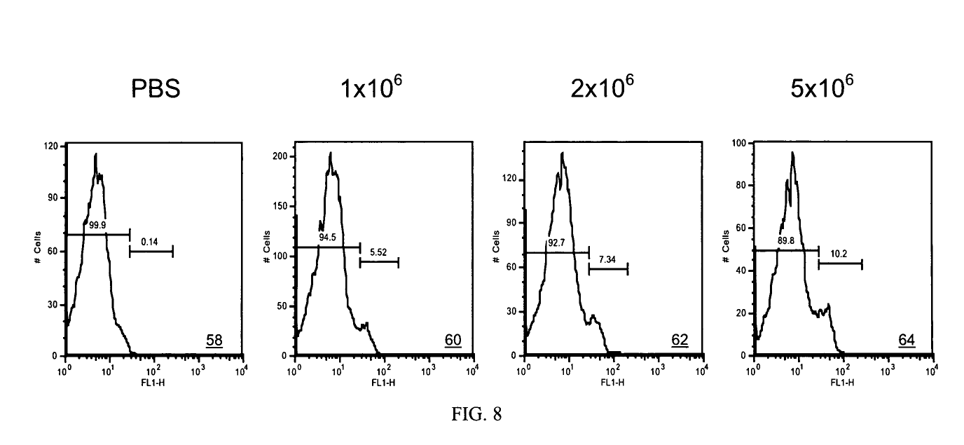

Next, the number of stained apoptotic tumor cells injected into mice was varied, and the percentage of DCs that showed uptake of apoptotic tumor cells was determined by flow cytometry. The results are presented in FIG. 8.

The histogram of graph 58 is a negative control wherein the injection contained phosphate buffered saline (PBS) only. The number associated with the bar on the left side of the histogram profile indicates the percentage of total DCs that have not uptaken apoptotic tumor cells. Conversely, the number associated with the bar on the right side of the histogram profile indicates the percentage of total DCs that have uptaken injected apoptotic tumor cells. In the negative control, 0.14% indicates the amount of background.

As indicated above graphs 60, 62 and 64, 1×10⁶, 2×10⁶ and 5×10⁶ apoptotic tumor cells were injected into mice, respectively, and the percentage of DCs that showed uptake of tumor cells was determined. When 1×10⁶ apoptotic cells were injected (graph 60) 5.52% of DCs showed uptake. Graph 60 shows that when 2×10⁶ tumor cells were injected 7.34% of DCs showed uptake, and graph 60 shows that 10.2% of DCs showed uptake when 5×10⁶ tumor cells were injected. As expected, the percentage of DCs that show uptake increases as the number injected apoptotic tumor cells increases.

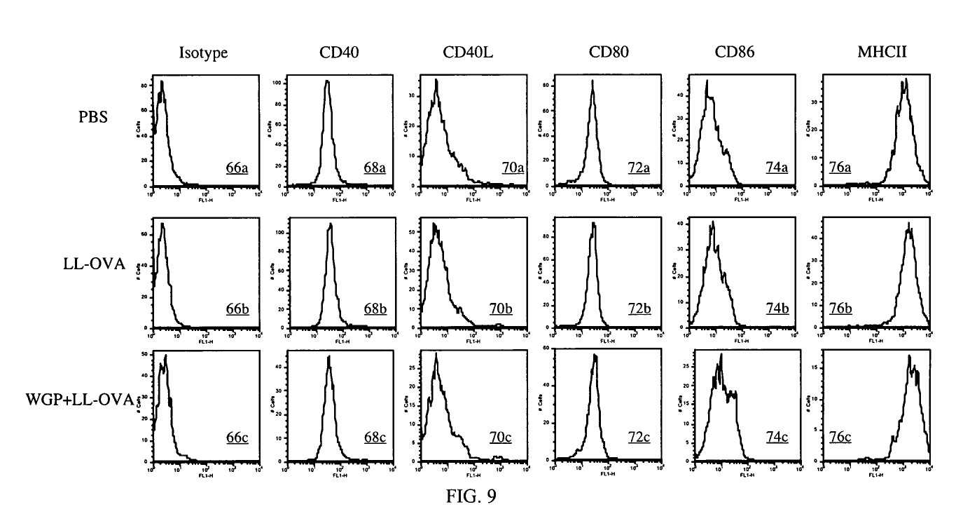

Then, the ability of particulate β-glucan to stimulate splenic DC maturation in vivo was tested. Mice were given either PBS, apoptotic OVA expressing lewis lung carcinoma cells (LL-OVA) or apoptotic LL-OVA+400 μg/day of particulate β-glucan for three days and surface marker expression of splenic DCs was determined by flow cytometry. The results are shown in FIG. 9.

Graphs 66a, 66b and 66c show histograms of isotype Ab controls for PBS-negative control mice, LL-OVA mice and LLC-OVA+particulate β-glucan mice, respectively. Similarly, graphs 68a-c, 70a-c, 72a-c and 74a-c include histograms representing CD40, CD80, CD86 and MHCII expression, respectively.

CD86 and MHCII are the most relevant cytokines related to adaptive immunity. Accordingly, the histogram shift seen between graph 72b and 72c and between 74b and 74c indicate increased expression of these cytokines in the presence of particulate β-glucan. Thus, administration of about 25 μg/ml or more of particulate β-glucan results in increased expression of costimulatory molecules, which, in turn, results in increased antigen presenting capacity. The increased antigen presentation results in increased T cell expansion. These experiments have shown that particulate β-glucan enhances the adaptive immune system to increase its effectiveness against uncontrolled proliferating cells in diseases such as cancer.

Conversely, it has also come to light that specific dosages of zymosan, a cell wall preparation of S. cerevisiae that contains β-glucan, results in inhibitory cytokines that neutralize inflammatory cytokines. (Dillon et al., J. Clin. Invest. 116:916-928 (2006)). In experiments similar to those described above, administrations of zymosan at about 5 μg/ml or less resulted in increased expression of IL-10 and TGF-β. The secretion of these cytokines leads to immunological tolerance. This mechanism may likely lead to treatment of autoimmune disorders, which are characterized by chronic inflammation.

Taken together, these results identify a method and composition that utilizes particulate β-glucan for either enhancing the adaptive immune response or inducing immunological tolerance depending on dosage. While not being bound by theory, evidence supports a mechanism whereby signaling of DCs by β-glucan is through dectin-1 and/or toll-like receptor 2 (TLR2).

Dectin-1 and TLR2 are representative of a group of immune cell receptors called pathogen-recognition receptors (PRRs). PRRs recognize and interpret highly conserved ligands on various microbes to produce appropriate immune responses.

As described above, the present composition includes particulate β-glucan such as whole glucan particles purified from the cell walls of S. cerevisiae. The particulate β-glucan may be administered by any means including, for example, oral, parenteral or topical. The composition may also include an appropriate carrier.

In a representative method for preparing particulate β-glucan, a yeast culture is grown, typically, in a shake flask or fermenter. In one embodiment of bulk production, a culture of yeast is started and expanded stepwise through a shake flask culture into a 250-L scale production fermenter. The yeast are grown in a glucose-ammonium sulfate medium enriched with vitamins, such as folic acid, inositol, nicotinic acid, pantothenic acid (calcium and sodium salt), pyridoxine HCl and thymine HCl and trace metals from compounds such as ferric chloride, hexahydrate; zinc chloride; calcium chloride, dihydrate; molybdic acid; cupric sulfate, pentahydrate and boric acid. An antifoaming agent such as Antifoam 204 may also be added at a concentration of about 0.02%.

The production culture is maintained under glucose limitation in a fed batch mode. During seed fermentation, samples are taken periodically to measure the optical density of the culture before inoculating the production fermenter. During production fermentation, samples are also taken periodically to measure the optical density of the culture. At the end of fermentation, samples are taken to measure the optical density, the dry weight, and the microbial purity.

If desired, fermentation may be terminated by raising the pH of the culture to at least 11.5 or by centrifuging the culture to separate the cells from the growth medium. In addition, depending on the size and form of purified β-glucan that is desired, steps to disrupt or fragment the yeast cells may be carried out. Any known chemical, enzymatic or mechanical methods, or any combination thereof may be used to carry out disruption or fragmentation of the yeast cells.

The yeast cells containing the β-glucan are harvested. When producing bulk β-glucan, yeast cells are typically harvested using continuous-flow centrifugation.

Yeast cells are extracted utilizing one or more of an alkaline solution, a surfactant, or a combination thereof. A suitable alkaline solution is, for example, 0.1 M-5 M NaOH. Suitable surfactants include, for example, octylthioglucoside, Lubrol PX, Triton X-100, sodium lauryl sulfate (SDS), Nonidet P-40, Tween 20 and the like. Ionic (anionic, cationic, amphoteric) surfactants (e.g., alkyl sulfonates, benzalkonium chlorides, and the like) and nonionic surfactants (e.g., polyoxyethylene hydrogenated castor oils, polyoxyethylene sorbitol fatty acid esters, polyoxyethylene sorbitan fatty acid esters, polyoxyethylene glycerol fatty acid esters, polyethylene glycol fatty acid esters, polyoxyethylene alkyl phenyl ethers, and the like) may also be used. The concentration of surfactant will vary and depend, in part, on which surfactant is used. Yeast cell material may be extracted one or more times.

Extractions are usually carried out at temperatures between about 70° C. and about 90° C. Depending on the temperature, the reagents used and their concentrations, the duration of each extraction is between about 30 minutes and about 3 hours.

After each extraction, the solid phase containing the β-glucan is collected using centrifugation or continuous-flow centrifugation and resuspended for the subsequent step. The solubilized contaminants are removed in the liquid phase during the centrifugations, while the β-glucan remains in the insoluble cell wall material.

In one embodiment, four extractions are carried out. In the first extraction, harvested yeast cells are mixed with 1.0 M NaOH and heated to 90° C. for approximately 60 minutes. The second extraction is an alkaline/surfactant extraction whereby the insoluble material is resuspended in 0.1 M NaOH and about 0.5% to 0.6% Triton X-100 and heated to 90° C. for approximately 120 minutes. The third extraction is similar to the second extraction except that the concentration of Triton X-100 is about 0.05%, and the duration is shortened to about 60 minutes. In the fourth extraction, the insoluble material is resuspended in about 0.05% Triton-X 100 and heated to 75° C. for approximately 60 minutes.

The alkaline and/or surfactant extractions solubilize and remove some of the extraneous yeast cell materials. The alkaline solution hydrolyzes proteins, nucleic acids, mannans, and lipids. Surfactant enhances the removal of lipids, which provides an additional advantage yielding an improved β-glucan product.

The next step in the purification process is an acidic extraction shown, which removes glycogen. One or more acidic extractions are accomplished by adjusting the pH of the alkaline/surfactant extracted material to between about 5 and 9 and mixing the material in about 0.05 M to about 1.0 M acetic acid at a temperature between about 70° C. and 100° C. for approximately 30 minutes to about 12 hours.

In one embodiment, the insoluble material remaining after centrifugation of the alkaline/surfactant extraction is resuspended in water, and the pH of the solution is adjusted to about 7 with concentrated HCl. The material is mixed with enough glacial acetic acid to make a 0.1 M acetic acid solution, which is heated to 90° C. for approximately 5 hours.

Next, the insoluble material is washed. In a typical wash step, the material is mixed in purified water at about room temperature for a minimum of about 20 minutes. The water wash is carried out two times. The purified β-glucan product is then collected. Again, collection is typically carried out by centrifugation or continuous-flow centrifugation.

At this point, a purified, particulate β-glucan product is formed. The product may be in the form of whole glucan particles or any portion thereof, depending on the starting material. In addition, larger sized particles may be broken down into smaller particles. The range of product sizes includes β-glucan particles that have substantially retained in vivo morphology (whole glucan particles) down to submicron-size particles.

A second representative process for producing whole glucan particles involves the extraction and purification of the alkali-insoluble whole glucan particles from the yeast or fungal cell walls. This process yields a product, which maintains the morphological and structural properties of β-glucan as found in vivo.

The source of whole glucan can be yeast or other fungi, or any other source containing glucan having the properties described herein. In certain embodiments, yeast cells are a preferred source of glucans. The yeast strains employed in the present process can be any strain of yeast.

Generally, the above procedure can be used to prepare and isolate other mutant yeast strains with other parent strains as starting material. Additionally, mutagens can be employed to induce the mutations, for example, chemical mutagens, irradiation, or other DNA and recombinant manipulations. Other selection or screening techniques may be similarly employed.

The yeast cells may be produced by methods known in the art. Typical growth media comprise, for example, glucose, peptone and yeast extract. The yeast cells may be harvested and separated from the growth medium by methods typically applied to separate the biomass from the liquid medium. Such methods typically employ a solid-liquid separation process such as filtration or centrifugation. In the present process, the cells may be harvested in the mid- to late-logarithmic phase of growth, to minimize the amount of glycogen and chitin in the yeast cells. Glycogen, chitin and protein can affect the biological and hydrodynamic properties of the whole glucan particles.

Preparation of whole glucan particles involves treating the yeast with an aqueous alkaline solution at a suitable concentration to solubilize a portion of the yeast and form alkali-hydroxide insoluble whole glucan particles having primarily linkages. The alkali generally employed is an alkali-metal hydroxide, such as sodium or potassium hydroxide or an equivalent. The starting material can comprise yeast separated from the growth medium. It is more difficult to control consumption of the aqueous hydroxide reactants and the concentration of reactants in the preferred ranges when starting with yeast compositions that are less concentrated. The yeast cells are treated in the aqueous hydroxide solution. The intracellular components and mannoprotein portion of the yeast cells are solubilized in the aqueous hydroxide solution, leaving insoluble cell wall material, which is substantially devoid of protein and having a substantially unaltered three dimensional matrix of linked glucan. The preferred conditions of performing this step result in the mannan component of the cell wall being dissolved in the aqueous hydroxide solution. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

In certain embodiments, the aqueous hydroxide digestion step is carried out in a hydroxide solution having an initial normality of from about 0.1 to about 10.0. Typical hydroxide solutions include hydroxides of the alkali metal group and alkaline earth metals of the Periodic Table. Suitable aqueous hydroxide solutions are of sodium and potassium. The digestion can be carried out at a temperature of from about 20° C. to about to about 121° C. with lower temperatures requiring longer digestion times. When sodium hydroxide is used as the aqueous hydroxide, the temperature can be from about 80° C. to about 100° C., and the solution has a normality of from about 0.75 to about 1.5. The hydroxide added is in excess of the amount required, thus, no subsequent additions are necessary.

Generally from about 10 to about 500 grams of dry yeast per liter of hydroxide solution is used. In certain embodiments, the aqueous hydroxide digestion step is carried out by a series of contacting steps so that the amount of residual contaminants such as proteins are less than if only one contacting step is utilized. In certain embodiments, it is desirable to remove substantially all of the protein material from the cell. Such removal is carried out to such an extent that less than one percent of the protein remains with the insoluble cell wall glucan particles. Additional extraction steps are preferably carried out in a mild acid solution having a pH of from about 2.0 to about 6.0. Typical mild acid solutions include hydrochloric acid, sodium chloride adjusted to the required pH with hydrochloric acid and acetate buffers. Other typical mild acid solutions are in sulfuric acid and acetic acid in a suitable buffer. This extraction step is preferably carried out at a temperature of from about 20° C. to about 100° C. The glucan particles can be, if necessary or desired, subjected to further washings and extraction to reduce the protein and contaminant levels. After processing the product pH can be adjusted to a range of about 6.0 to about 7.8.

The whole glucan particles can be further processed and/or further purified, as desired. For example, the glucan can be dried to a fine powder (e.g., by drying in an oven); or can be treated with organic solvents (e.g., alcohols, ether, acetone, methyl ethyl ketone, chloroform) to remove any traces or organic-soluble material, or retreated with hydroxide solution, to remove additional proteins or other impurities that may be present.

Whole glucan particles, as produced above, may be modified by chemical treatment to decrease the number of β(1-6) linkages and, thus, change the properties of the β-glucan. According to a first chemical treatment, the whole glucan particles can be treated with an acid to decrease the amount of hydrodynamic properties of said glucans as evidenced by an increase in the viscosity of aqueous solutions of these modified glucans. A process for preparing altered whole glucan particles by treating the glucan particles with an acid, for a suitable period of time to alter the β(1-6) linkages may be used. Acetic acid is preferred, due to its mild acidity, ease of handling, low toxicity, low cost and availability, but other acids may be used. Generally these acids should be mild enough to limit hydrolysis of the β(1-3) linkages. The treatment is carried out under conditions to substantially only affect the β(1-6) linked glucans. In certain embodiments, the acid treatment is carried out with a liquid consisting essentially of acetic acid, or any dilutions thereof (typical diluents can be organic solvents or inorganic acid solutions). The treatment is carried out at a temperature of from about 20° C. to about 100° C. to remove from about 3 to about 20 percent by weight of acid soluble material based on total weight of the whole glucan particles before treatment. In other embodiments, the extent of removal is from about 3 to about 4 percent by weight. Certain compositions formed demonstrate altered hydrodynamic properties and an enhancement in viscosity after treatment.

According to a second chemical treatment, the whole glucan particles are treated with an enzyme or an acid to change the amount of β(1-3) linkages. Depending on the yeast strain, enzyme treatment either causes a decrease in the viscosity or an increase in viscosity, but in general, alters the chemical and hydrodynamic properties of the resulting glucans. The treatment is with a β(1-3) glucanase enzyme, such as laminarinase, for altering the β(1-3) linkages and the hydrodynamic properties of the whole glucan particles in aqueous suspensions. The enzyme treatment can be carried out in an aqueous solution having a concentration of glucan of from about 0.1 to about 10.0 grams per liter. Any hydrolytic glucanase enzyme can be used, such as laminarinase, which is effective and readily available. The time of incubation may vary depending on the concentration of whole glucan particles and glucanase enzyme. The β(1-3) linkages are resistant to weak acids, such as acetic acid. Treatment with strong or concentrated acids, such as hydrochloric acid (HCl), sulfuric acid (HSO) or formic acid, hydrolyzes β(1-3) linkages. The acid treatment can be carried out in an aqueous solution having a concentration of glucan from about 0.1 to about 10.0 grams per liter. The time of acid treatment may vary depending upon the concentration of whole glucan particles and acid. Acid hydrolysis can be carried out at a temperature of from about 20° C. to about 100° C.

By controlling the incubation time, it is possible to control the chemical and hydrodynamic properties of the resulting product. For example, the product viscosity can be precisely controlled for particular usage, as, for example, with a variety of food products.

A hydrodynamic parameter (K) of the final treated product having altered linkages is dependent on the treatment time according to the final formula:

K₁=−0.0021(time)+0.26

where time is in minutes; and
where time is less than one hour.
The parameter K₁ is directly related (proportional) to the relative viscosity. In the case of aqueous suspensions the relative viscosity is equal to the actual viscosity when the latter is measured in centipoise.

A process for preparing an aqueous slurry of glucan having a predetermined desired viscosity is provided. The slurry comprises glucan at a concentration that is a function of the predetermined desired viscosity according to the following approximate formula:

1/concentration=K₁×(1/log(relative viscosity))+K₂

### Where,

K₁=(shape factor)×(hydrodynamic volume); and
K₂=(hydrodynamic volume)/(maximum packing fraction).
The shape factor is an empirically determined value that describes the shape of the glucan matrix in its aqueous environment. The shape factor is a function of the length:width ratio of a particle and can be determined microscopically. The hydrodynamic volume is a measure of the volume a particle occupies when in suspension. This is an important parameter for glucan suspensions in that it indicates the high water holding capacity of glucan matrices. The maximum packing fraction can be described as the highest attainable volume fraction of glucans that can be packed into a unit volume of suspension.

Microparticulate β-glucan may also be used, and the starting material can be isolated from yeast cell walls by conventional methods known by those of ordinary skill in the art. The general method for the production of glucan from yeast involves extraction with alkali followed by extraction with acid (Hassid et al., Journal of the American Chemical Society, 63:295-298, 1941). Improved methods for isolating a purified water insoluble β-glucan extract are disclosed in U.S. Pat. No. 5,223,491, which is incorporated herein by reference in its entirety. Another method of producing whole glucan particles is disclosed in U.S. Pat. No. 4,992,540, which is incorporated herein by reference in its entirety. Methods for preparing microparticulate β-glucan are disclosed in U.S. Pat. No. 5,702,719, the disclosure of which is incorporated herein by reference in its entirety. Microparticulate β-glucan product can be obtained with the average particle size of about 1.0 micron or less or about 0.20 microns or less.

Microparticulate β-glucan particles can be reduced in size by mechanical means such as by blender, microfluidizer, or ball mill, for example. For example, particle size can be reduced using a blender having blunt blades, wherein the glucan mixture is blended for a sufficient amount of time, preferably several minutes, to completely grind the particles to the desired size without overheating the mixture. Another grinding method comprises grinding the glucan mixture in a ball mill with 10 mm stainless steel grinding balls. This latter grinding method is particularly preferred when a particle size of about 0.20 microns or less is desired.

Prior to grinding, the glucan mixture is preferably passed through a series of sieves, each successive sieve having a smaller mesh size than the former, with the final mesh size being about 80. The purpose of sieving the mixture is to separate the much larger and more course glucan particles from smaller particles (the pore size of an 80 mesh sieve is about 0.007 inches or 0.178 mm). The separated larger particles are then ground down as described above and re-sieved to a final mesh size of 80. The process of sieving and grinding is repeated until a final mesh size of 80 is obtained. The sieved particles are combined and ground down further, preferably for at least an hour, until the desired particle size is obtained, preferably about 1.0 micron or less, more preferably about 0.20 microns or less. Periodic samples of the fine grind glucan are taken during the grinding process and measured using a micrometer on a microscope.

Oral formulations suitable for use in the practice of the present invention include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. An additional formulation is for delivery intranasally. The compounds of the present invention may also be presented as a bolus, electuary, or paste.

Generally, formulations are prepared by uniformly mixing the active ingredient with liquid carriers or finely divided solid carriers or both, and then if necessary shaping the product. A pharmaceutical carrier is selected on the basis of the chosen route of administration and standard pharmaceutical practice. Each carrier must be “acceptable” in the sense of being compatible with the other ingredients of the formulation and not injurious to the subject. This carrier can be a solid or liquid and the type is generally chosen based on the type of administration being used. Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents.

Preferred carriers are edible oils, for example, corn or canola oils. Polyethylene glycols, e.g., PEG, are also preferred carriers. The formulations for oral administration may comprise a non-toxic, pharmaceutically acceptable, inert carrier such as lactose, starch, sucrose, glucose, methyl cellulose, magnesium stearate, dicalcium phosphate, calcium sulfate, mannitol, sorbitol, cyclodextrin, cyclodextrin derivatives, or the like.

The composition, if desired, can also contain minor amounts of wetting or emulsifying agents, or pH buffering agents. The composition can be a liquid solution, suspension, emulsion, tablet, pill, capsule, sustained release formulation, or powder.

The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. Nebulized formulation for inhalation can include sodium chloride, sodium saccharine or sorbitani trioleas, whereas inhalation via compressed carbonated formulation in a puffer can include 1,1,1,2-tetrafluoroethanum, monofluorotrichloromethanum tetrafluorodichloroaethanum or difluorodichloromethanum.

Capsule or tablets can be easily formulated and can be made easy to swallow or chew. Tablets may contain suitable carriers, binders, lubricants, diluents, disintegrating agents, coloring agents, flavoring agents, flow-inducing agents, or melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, or the like.

Lubricants used in these dosage forms include sodium oleate, sodium stearate, magnesium stearate, sodium benzoate, sodium acetate, sodium chloride, or the like. Disintegrators include, for example, starch, methyl cellulose, agar, bentonite, xanthan gum, or the like. Molded tablets may be made by molding in a suitable machine a mixture of the powdered active ingredient moistened with an inert liquid diluent.

The tablets may optionally be coated or scored and may be formulated so as to provide slow- or controlled-release of the active ingredient. Tablets may also optionally be provided with an enteric coating to provide release in parts of the gut other than the stomach.

Exemplary pharmaceutically acceptable carriers and excipients that may be used to formulate oral dosage forms of the present invention are described in U.S. Pat. No. 3,903,297 to Robert, issued Sep. 2, 1975, incorporated by reference herein in its entirety. Techniques and compositions for making dosage forms useful in the present invention are described in the following references: 7 Modem Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injections solutions and suspensions may be prepared from sterile powders, granules and tablets of the kind previously described. Parenteral and intravenous forms may also include minerals and other materials to make them compatible with the type of injection or delivery system chosen.

While this invention has been shown and described with references to particular embodiments, it will be understood by those skilled in the art that various changes in form and detail may be made therein without departing from the spirit and scope of the invention encompassed by the appended claims.

1. A method of modulating cytokine production in an animal to treat a disease, the method comprising:
administering particulate β-glucan to the animal at one of a low concentration or a high concentration;
wherein the high concentration increases secretion of inflammatory cytokines and the low concentration increases secretion of anti-inflammatory cytokines.
